(19) United States
(12) Patent Application Publication
(10) Pub. No.: US 2015/0148620 A1
(43) Pub. Date: May 28, 2015

(54) MONITORING SYSTEM

(71) Applicant: iBio Products LLC, Kannapolis, NC (US)

(72) Inventors: Farid Roy, Mooresville, NC (US); Laila Roy, Mooresville, NC (US); Nadir Noorani, Concord, NC (US); Salim Punjani, Atlanta, NC (US); Shaheen Noorani, Concord, NC (US); Shahina Punjani, Flowery Branch, GA (US)

(21) Appl. No.: 14/542,628

(22) Filed: Nov. 16, 2014

Related U.S. Application Data
(60) Provisional application No. 61/908,128, filed on Nov. 24, 2013.

(51) Int. Cl. A61B 5/00; A61B 5/1455; A61B 5/145; A61B 5/0205; A61B 5/11

(52) U.S. Cl. CPC A61B 5/7282; A61B 5/02055; A61B 5/1118; A61B 5/14532; A61B 5/14551; A61B 5/7275; A61B 5/7405; A61B 5/742; A61B 5/021
USPC 600/301

(57) ABSTRACT

A notification criteria corresponding to vital signs of a mammal is statically or dynamically determined. The detected and/or measured values for the vital signs are continuously and/or periodically compared to the notification criteria to determine if the values match the notification criteria. If the values match the notification criteria, a notification is transmitted or presented.

### TECHNICAL FIELD

The present disclosure relates to a monitoring system. In particular, the present disclosure relates to monitoring a mammal's vital signs and providing related data and/or notifications.

### BACKGROUND

Sensors have emerged as a fast-growing market for the collection of data. The data collected by sensors includes data related to a mammal's vital signs which may be useful for diagnosing the mammal's health. A mammal is a well-defined term well known to a person of ordinary skill in the art. Examples of mammals include, but are not limited to, humans, dogs, and horses.

Vital signs as referred to herein include any measurements of a mammal that can be measured and/or detected. Examples of vital signs include body temperature, respiration rate, pulse rate, pulse strength, heart beat rhythm, blood pressure, blood glucose level, oxygen saturation (e.g., via pulse oximetry), consciousness state (e.g., via Glasgow coma scale), inflammation, and perspiration rate.

The approaches described in this section are approaches that could be pursued, but not necessarily approaches that have been previously conceived or pursued. Therefore, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for triggering a notification;

FIG. 3 illustrates an example in accordance with one or more embodiments.

### DETAILED DESCRIPTION

In the following description, for the purposes of explanation, numerous specific details are set forth in order to provide a thorough understanding. One or more embodiments may be practiced without these specific details. Features described in one embodiment may be combined with features described in a different embodiment. In some examples, well-known structures and devices are described with reference to a block diagram form in order to avoid unnecessarily obscuring the present invention. The detailed description includes the following sections:
1. GENERAL OVERVIEW
2. ARCHITECTURAL OVERVIEW
3. TRIGGERING A NOTIFICATION
3.1 DETERMINING A NOTIFICATION CRITERIA
   3.2 OBTAINING VALUES CORRESPONDING TO VITAL SIGNS
   3.3 COMPARING VITAL SIGNS TO NOTIFICATION CRITERIA
   3.4 PRESENTING OR TRANSMITTING A NOTIFICATION
4. EXAMPLE EMBODIMENT
5. MISCELLANEOUS; EXTENSIONS

### 1. General Overview

In an embodiment, a notification criteria corresponding to vital signs of a mammal is statically or dynamically determined. The detected and/or measured values for the vital signs are continuously and/or periodically compared to the notification criteria to determine if the values match the notification criteria. If the values match the notification criteria, a notification is transmitted or presented.

2. Architechtural Overview

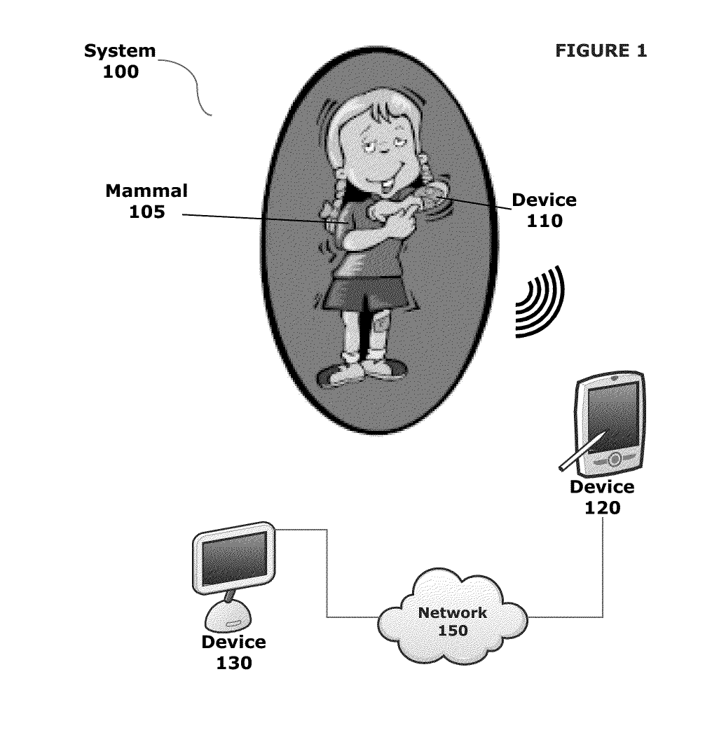

FIG. 1 illustrates a system (100) in accordance with one or more embodiments. Although a specific system is described, other embodiments are applicable to any system that can be used to perform the functionality described herein. Additional or alternate components may be included that perform functions described herein. Components described herein may be altogether omitted in one or more embodiments. One or more components described within system (100) may be combined together in a single device.

Components of the system (100) are connected by, without limitation, a network such as a Local Area Network (LAN), Wide Area Network (WAN), the Internet, Intranet, Extranet, and/or satellite links. Any number of devices connected within the system (100) may be directly connected to each other through wired and/or wireless communication segments (e.g., via Network (150)). In one example, devices within system (100) are connected via a direct wireless connection such a Bluetooth connection, a Near Field Communication (NFC) connection, and/or a direct Wi-Fi connection.

In an embodiment, device (110), device (120), and device (130) generally represent any device that includes a hardware processor. Examples of devices in accordance with one or more embodiments include, but are not limited to, a desktop, a laptop, a tablet, a phablet, a cellular phone, a smart phone, a Personal Digital Assistant (PDA), and a kiosk.

In an embodiment, device (110) is a device configured to measure or detect one or more vital signs from mammal (105). Mammal (105) is illustrated as a human user, however embodiments are equally applicable to other mammals.

In an embodiment, device (110) measures or detects one or more vital signs via sensors. Vital signs as referred to herein may include, but are not limited to, body temperature, respiration rate, pulse rate, pulse strength, heart beat rhythm, blood pressure, blood glucose level, weight, oxygen saturation (e.g., via pulse oximetry), consciousness state (e.g., via Glasgow coma scale), inflammation, and perspiration rate. Examples of sensors include, but are not limited to, thermometers, accelerometers, auxanometer, capacitive displacement sensor, gyroscopic sensor, a linear encoder, a position sensor, and a velocity receiver.

In an embodiment, the one or more sensors of device (110) are configured to detect an activity level of a mammal (e.g., resting, moving slowly, moving quickly etc.). Embodiments are applicable to any type of sensor which collect data related to an activity level of a mammal (105). For purposes of explanation, only a limited set of applicable sensors are described herein.

In an embodiment, the data collected by sensors indicates an actual activity (e.g., sleeping, walking, swimming, and running) of a mammal. In an example, data collected by sensors indicates a lack of movement and snoring sounds. The data may be used to determine that the mammal (105) is sleeping. In another example, data may indicate that the sensor is submerged in liquid with a high chlorine content indicating that the mammal is in a swimming pool.

Sensors may be located outside of a mammal's body (e.g., strapped on and/or stuck on), or inside of a mammal's body (e.g. implanted underneath the skin or dispensed in the blood). In an example, a sensor (e.g., Thermometer) may measure body temperature is taken orally, rectally, via axillary methods (e.g., armpit), via the groin area, via an ear, via skin (e.g., temple), or via any other medically accepted method.

In an embodiment, sensors collect data related to a mammal's environment including, but not limited to, ambient temperature, humidity, air pressure, presence of smoke, presence of bacteria, presence of blood, and presence of a virus.

In an embodiment, data related to a mammal's activity and/or environment may be collected through alternate sources of information related to the mammal. For example, device (120) access's a user Jane's schedule to determine that Jane is scheduled to work out at the gym from 7 pm to 8 pm. Based on the schedule, Jane's activity from 7 pm to 8 pm is determined to be “working out”. In another example, a location of Joe's cell phone is used to determine that Joe is currently located in the zip code 85023. Based on Joe's current zip code of 85023, an ambient temperature search is executed that the current temperature in zip code 85023 (corresponding to Phoenix, Arizona) is 115 degrees. In another example, Jim's email account is analyzed to determine that Jim will be on a flight from 3 pm to 7 pm on Jan. 11, 2015.

In an embodiment, the data storage of any device (e.g., device (110), device (120), and device (130)) includes a fast read-write memory for storing programs and data during the execution of an operating system and a hierarchy of persistent memory such as ROM, EPROM, and Flash memory for storing instructions and data needed for the startup and/or operations of device (110). In an embodiment, the I/O interface of device (110) corresponds to one or more components used for communicating with other devices via wired or wireless segments. The I/O interface may include a wired network interface such as an IEEE 802.3 Ethernet interface and/or a wireless interface such as an IEEE 802.11 Wi-Fi® interface.

In an embodiment, a processor is coupled to the data storage and the I/O interface. The processor may be any processing device including, but not limited to a MIPS-class processor, a microprocessor, a digital signal processor, an application specific integrated circuit, a microcontroller, a state machine, or any type of programmable logic array.

### 3. Triggering a Notification

In one or more embodiments, data collected by sensors regarding a mammal's current vital signs is compared to a notification criteria. If the data matches the notification criteria, a notification is triggered. The notification may be transmitted or presented by a device.

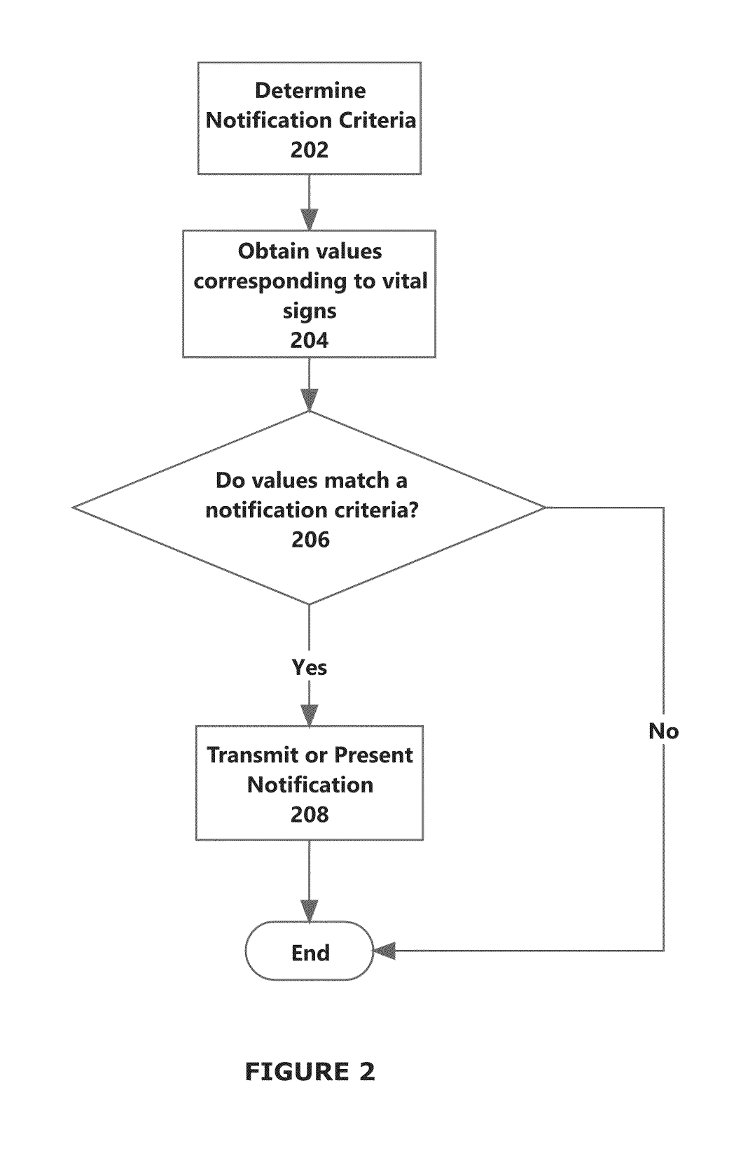

FIG. 2 illustrates an example set of operations for triggering a notification. Operations for triggering a notification, as described herein with reference to FIG. 2, may be omitted, rearranged, or modified. Furthermore, operations may be added or performed by different components or devices. Accordingly, the specific set or sequence of operations should not be construed as limiting the scope of any of the embodiments.

### 3.1 Determining a Notification Criteria

In an embodiment, a notification criteria associated with one or more current vital signs of a mammal is determined (Operation 202). The notification criteria may be received by a device or determined by the device itself based on data available to the device (for example, data collected by a sensor).

In an embodiment, the notification criteria associated with one or more current vital signs is statically determined. For example, a medical authority defines an unacceptable glucose range which requires immediate attention from a doctor. Based on the information from the medical authority, the notification criteria is hard coded for presenting or transmitting notifications when a user's glucose level falls within the unacceptable glucose range.

In an embodiment, the notification criteria is defined by a user. For example, a parent provides user input indicating that the parent is to be notified if a child's body temperature is above 100 degrees. The parent may provide user input into any device communicatively coupled to the system or directly to a device collecting the data (e.g., device (110) in FIG. 1).

In an embodiment, the notification criteria associated with one or more current vital signs of a mammal is dynamically determined based on a current activity of a mammal. In an example, a user Shyna is wearing an activity tracker on her wrist. The data collected by the activity tracker indicates that Shyna is running. Based on the known activity of running and the statistical information on heart rates for users that are running, an acceptable heart rate range is dynamically determined for Shyna. Any heart rate values that are outside of the acceptable heart rate range are included in the notification criteria as requiring the generation of a notification.

In another example, a user Salim is determined to be sleeping based on a 3 am time of day, an identified location of Salim inside his bedroom, and a consciousness state. Based on Salim's known activity of sleeping and the statistical information of body temperature, an acceptable body temperature range of 98.4 to 98.8 is determined. Body temperature values higher than 98.8 and lower than 98.4 are defined as unacceptable and included in a notification criteria as requiring the generation of a notification.

In an embodiment, the notification criteria associated with one or more current vital signs of a mammal is dynamically determined based on an environment in which the mammal is located. In an example, a user named Nadir is scheduled to be watching a football game in a stadium in Santa Clara, Calif. at night during November. A device determines that the ambient temperature in the stadium will be 55 degrees. Based on the ambient temperature in the stadium, Nadir's body temperature is expected to be lower than normal. Based on an ambient temperature of 55 degrees, the device modifies a first notification criteria of alerting for detected body temperatures of below 98.2 degrees to a second notification criteria of alerting for detected body temperatures of below 97.6 degrees.

In an embodiment, the notification criteria associated with one or more current vital signs of a particular mammal is dynamically determined based on the other mammals with which the particular mammal is interacting. For example, a user Laila is known to be spending the day with her mother-in-law which typically increases a stress level for Laila. Based on the expected increase in stress level, a threshold value for the blood pressure which is used to trigger a notification is increased to allow for the temporary expected increase in blood pressure without the triggering of a notification.

In an embodiment, the notification criteria associated with one or more current vital signs of a mammal is dynamically determined based on attributes of the mammal being monitored. For example, medical authorities may publish various ranges of acceptable pulse strength values for different age groups. The system identifies the user based on user input or by detecting information about the user (e.g., fingerprint) and querying a database using the detected information. Once the user is identified, the system then determines the information relevant to determining the notification criteria (e.g., age, gender, health conditions, risks, family health history, or any other information to be used for determining the notification criteria). In some cases, the relevant information may be directly entered. For example, a user's history of family heart issues is entered into the system via user input and the system dynamically determines the applicable notification criteria based on the family history of heart issues.

In an embodiment, the notification criteria associated with one or more current vital signs of a mammal is dynamically determined based on a current location of the mammal being monitored. In an example, a user Farid is determined to be hiking at high altitudes in the Himalayas. Users in the hiking in the Himalayas statistically have a higher respiration rate due to the low levels of oxygen in the air. As a result, Farid's a threshold value of respiration rate in the notification criteria is increased so that an alert is not triggered with the expected increase of the respiration rate.

In an embodiment, the notification criteria associated with one or more current vital signs of a particular mammal is dynamically determined based on how the values of the vital signs are being measured and/or detected. In an example, a child's body temperature reading measured via the child's armpit is up to one degree cooler than the child's temperature measured orally. In an embodiment, the system determines where the temperature is being measured and adjusts the notification criteria accordingly.

In an embodiment, the notification criteria associated with the current vital signs of a mammal being monitored is dynamically determined based on the vital signs of other mammals. In an example, when a first child is determined to have the flu, the first child's sibling, a second child has a higher chance of having the flu. As a result the notification criteria is re-calibrated to be very sensitive and immediately provide alerts at the smallest abnormalities/fluctuations in the second child's vital signs. In another example, a user Shaheen is at a theme park with her friends who are determined to be riding the same rides as Shaheen. When all friends' heart rate increase (e.g., as a result of riding the roller coaster), the notification criteria does not indicate that a notification is to be presented or transmitted. However, if Shaheen's heart rate increases outside of acceptable levels without her friends' heart rate increasing, then the notification criteria indicates that a notification should be presented or transmitted.

In an embodiment, the notification criteria may be dynamically modified to begin tracking or stop tracking of vital signs. In an example, if people in a town are known to be contracting a virus, a monitoring of chemicals related to that virus may be initiated by the system for other people in the same town.

In an embodiment, the notification criteria may be dynamically determined based on a distance of the mammal being monitored to other mammals or to particular locations. For example, the alert levels for a child's detected vital signs may be dynamically selected based on whether or not the child's mother is close to the child or away from the child. In another example, the alert levels for a senior citizens detected vital signs may be dynamically selected based on a distance of the senior citizen to the closest hospital.

### 3.2 Obtaining Values Corresponding to Vital Signs

In an embodiment, values corresponding to a mammal's vital signs are obtained (Operation 204). The values corresponding to a mammal's vital signs are obtained in real time as they are being detected or measured by sensors. For example, a single device performing monitoring and notification operations is attached to a user. The single device measures a user's pulse rate via sensors periodically (e.g., every minute or other frequency) and/or continuously.

In another example, a notification device is implemented separately from a monitoring device. The monitoring device collects values corresponding to the vital signs of a mammal. The notification device receives information periodically or continuously from the monitoring device either via a direct connection or via an indirect connection (i.e., through intermediate devices). The notification device and monitoring device may communicate via a push mechanism where data transfer is initiated by the monitoring device or via a pull mechanism where the notification device queries for the data from the monitoring device.

In an embodiment, the frequency for obtaining the values corresponding to the vital signs of a user are dynamically determined. In an example, a frequency is increased when a user is known to be sick, at risk for a particular medical diagnosis and/or has any other health related conditions. In another example, a frequency is increased when a user is participating in a high-risk activity such as travelling to a high disease-risk country or participating in a dangerous sport.

Obtaining values for current vital signs may include obtaining the instantaneous values or values corresponding to a period of time, e.g. the last five minutes, the last twenty-four hours.

In an embodiment, values corresponding to the vital signs of a user are obtained by a notification device from a monitoring device when the values meet the notification criteria. In an example, the monitoring device compares the values corresponding to the vital signs to a notification criteria. If the values meet the notification criteria, the data is forwarded to the separately implemented notification device.

### 3.3 Comparing Vital Signs to Notification Criteria

In an embodiment, a determination is made whether the values corresponding to a mammal's vital signs match a notification criteria (Operation 206). Determining whether the values match the notification criteria includes comparing the values to static notification criteria and/or dynamically determined notification criteria.

In an embodiment, determining whether the values match a notification criteria includes comparing the values to one or more threshold values. For example, a current body temperature of a particular user is compared to an acceptable range of body temperatures for users in general or compared to an acceptable range of body temperatures for users with the attributes of the particular user.

In an embodiment, determining whether the values match a notification criteria include comparing the values to multiple thresholds indicated in the notification criteria where each threshold corresponds to a different notification action. In an example, a body temperature of a user exceeding a first threshold of hundred degrees is associated with a notification action of alerting the user's mother. A body temperature of the user exceeding a second threshold of a hundred and three degrees is associated with a notification action of alerting emergency medical services.

In an embodiment, determining whether the values corresponding to a mammal's vital signs match a notification criteria include comparing a pattern of the detected/measured values to patterns identified in the notification criteria. For example, values for body temperature in the past twenty-four hours are compared to a notification criteria which indicates a pattern of increasing and decreasing body temperatures. In the example, the body temperature measurements for a user have switched back and forth between (a) increasing above a particular threshold and (b) decreasing to normal temperatures at least three times in twenty-four hours. Such a back and forth switch is indicative of a gall bladder infection. The notification criteria indicates that a notification must be presented or transmitted when such a back and forth switching pattern is detected.

In another example, a notification criteria defines an acceptable level of increase in heart rate (for example, as a result of exercising). The measured heart rate readings of a user compared to the acceptable level of increase in heart rate. Specifically, a rate of change in the measured heart rate readings is compared to an acceptable rate of change in heart rate readings. If the rate of change in the measured heart rate readings exceed the acceptable rate of change in heart rate readings, then the values match the notification criteria.

Other examples include comparing values for vital signs to patterns related to other users (e.g., users in the same vicinity, users with the same characteristics, users in a same group, family members, etc.). Notification criteria may include any pattern defined by a user (e.g., a parent, a nurse, a doctor, an administrator, etc.). Various examples of notification criteria patterns are described in section 3.1 titled “Determining A Notification Criteria.”

In an embodiment, different vital sign values may be compared to respective notification criteria at different frequencies. For example, values related to blood glucose levels may be compared to a respective notification criteria every ten minutes whereas values related to heart rate may be compared to a respective notification criteria every thirty seconds.

In an embodiment, a comparison of values to notification criteria may be triggered by a user. For example, a mother or a doctor may trigger the comparison of values for the vital signs of a child to a notification criteria. The most recently collected values may be used, or a new set of values may be collected in response to the trigger. The values are then compared to the notification criteria to determine if the values match the notification criteria.

If the values corresponding to the vital signs do not match the notification criteria, the evaluation cycle is completed. The process may restart at Operation 202 or at Operation 204. The notification criteria may be determined again with each evaluation cycle or a previously determined notification criteria may be used for the evaluation of new values in a next cycle.

### 3.4 Presenting or Transmitting a Notification

In an embodiment, if values corresponding to vital signs match a notification criteria, a notification is transmitted or presented (Operation 208). When multiple notification types and/or methods are available, a notification type and/or method is first selected.

In an embodiment, selecting notification types and/or methods include selecting a type of notification such as an audible notification, a visual notification, and/or a combination thereof. Notifications may also include notifying a user via smell (e.g., dispensing a particular chemical with a particular smell), touch (e.g., vibrating a beeper attached to a user), or taste.

In an embodiment, selecting notification types and/or methods include selecting recipients of the notification. The recipients of the notification may be statically defined or dynamically determined based on the detected/measured values corresponding to the vital signs. In an example, a child's father is always notified and a child's doctor is only notified for serious health hazards. In another example, a sequence of notifications is implemented. An adult user's sibling is notified first. If the sibling fails to acknowledge receipt of the notification, then a doctor is notified.

In an embodiment, selecting notification types and/or methods includes selecting a form of communication. For example, the system may be configured to notify via text message, email, a phone call, a page, a tweet, a social networking post, and/or through any other available means of communication.

In an embodiment, any Application Programming Interface (API) may be used by a monitoring device and/or a notification device to present, transmit, and/or obtain notifications. In one example, a Java object stores all data collected from sensors monitoring the vital signs of a user. The Java object is associated with an evaluation operation allows for comparison of the data to a notification criteria to evaluate the values and generate necessary notifications. The system exposes the API to medical systems accessible by a doctor who can execute operation on demand.

In an embodiment, selecting notification types and/or methods is based on location data related to the monitored user or location data related to a location of other entities in relation to the monitored user. In an example, an emergency service provider closest to the monitored user is determined in real-time in response to determining that values corresponding to vital signs match a high-alert notification criteria. In another example, a family member closest in distance to the monitored user is selected for notification.

In an embodiment, selecting notification types and/or methods are dynamically selected based on the values corresponding to the vital signs of the monitored mammal. In an example, if a respiration rate of a pet stops completely, then all of the lights in a household may be configured to flash on and off. If the respiration rate of a pet is lowered (e.g., pet is coughing), beeps may be sounded on an intercom system.

### 4. Example Embodiment

As noted above, embodiments and examples described herein are provided for illustrative purposes only and do not limit the scope of other embodiments described herein.

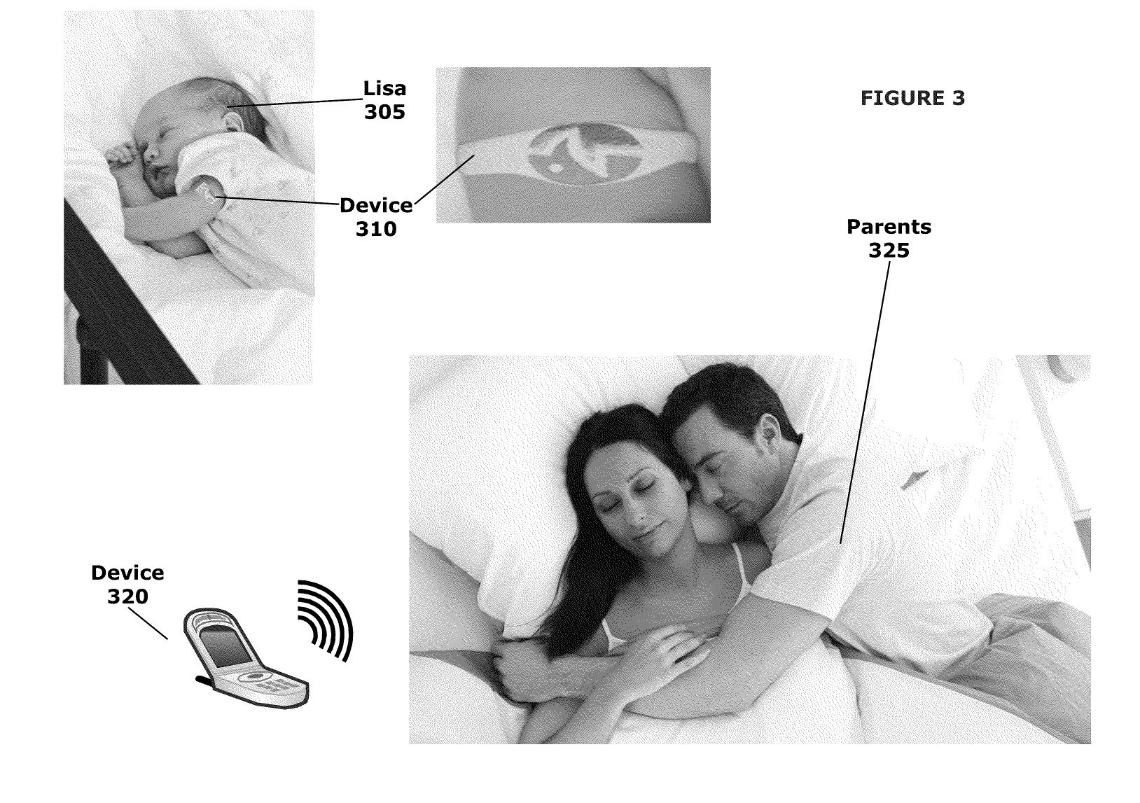

In one example illustrated in FIG. 3, a child Lisa (305) is not feeling well. Lisa's parents (325) are worried Lisa will catch a fever. Lisa's parents (325) have attached a monitoring device (310) to Lisa's arm for monitoring body temperature. A magnified version of the monitoring device (310) is included in FIG. 3. Monitoring device (310) continually measures Lisa's body temperature while Lisa (305) is asleep. Furthermore, monitoring device (310) continually transmits the collected values for body temperature to Lisa's parents' cell phone, notification device (320). Notification device (320) is executing a mobile application which receives the values corresponding to Lisa's body temperature. Lisa's parents have configured the mobile application executing notification device (320) such that if a body temperature reading over 99 degrees (notification criteria) is received from monitoring device (310), the mobile application will trigger an alert. The triggering of the alert by the mobile application will cause notification device (320) to beep loudly and flash lights that would wake up Lisa's parents (325) so that they can attend to Lisa (305) and help reduce her fever. If the mobile application does not receive any temperature reading over 99 degrees from monitoring device (310), the mobile application will not trigger any alert, allowing Lisa's parents (325) to peacefully sleep while Lisa also sleeps in her own crib. The mobile application executing on notification device (320) is further configured to immediately alert Lisa's doctor's cell phone (not shown) if Lisa's body temperature reading exceeds one hundred and one degrees.

### 5. Miscellaneous; Extensions

Embodiments are directed to a system with one or more devices that include a hardware processor and that are configured to perform any of the operations described herein and/or recited in any of the claims below.

In an embodiment, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

1. A non-transitory computer readable medium comprising instructions which, when executed by one or more hardware processors, causes performance of operations comprising:
identifying one or more current attributes associated with a mammal;
dynamically determining a notification criteria associated with one or more current vital signs of the mammal based on the one or more current attributes associated with the mammal;
receiving a plurality of values corresponding to the one or more current vital signs for a mammal;
determining that one or more values in the plurality of values match the dynamically determined notification criteria;
responsive to determining that the one or more values in the plurality of values match the dynamically determined notification criteria: transmitting or presenting a notification.

2. The medium of claim 1, wherein dynamically determining the notification criteria based on the one or more current attributes associated with the mammal comprises dynamically determining the notification criteria based on a current activity of the mammal.

3. The medium of claim 1, wherein dynamically determining the notification criteria based on the one or more current attributes associated with the mammal comprises dynamically determining the notification criteria based on an environment in which the mammal is currently located.

4. The medium of claim 1, wherein dynamically determining the notification criteria based on the one or more current attributes associated with the mammal comprises dynamically determining the notification criteria based on current vital signs associated with one or more other mammals.

5. The medium of claim 1, wherein dynamically determining the notification criteria based on the one or more current attributes associated with the mammal comprises dynamically determining the notification criteria based on current trends in vital signs associated with one or more other mammals.

6. The medium of claim 1, wherein dynamically determining the notification criteria based on the one or more current attributes associated with the mammal comprises dynamically determining the notification criteria based on a distance of the mammal from a second mammal.

7. The medium of claim 1, wherein dynamically determining the notification criteria based on the one or more current attributes associated with the mammal comprises dynamically determining the notification criteria based on a distance of the mammal from a medical facility.

8. A non-transitory computer readable medium comprising instructions which, when executed by one or more hardware processors causes performance of operations comprising:
receiving, by a first device from a second device, a plurality of values corresponding to one or more current vital signs for a mammal;
determining, by the first device, that at least one value in the received plurality of values matches a particular notification criteria;
responsive to determining that the at least one value matches the particular notification criteria, presenting or transmitting a notification.

9. The medium of claim 8, wherein the plurality of values are received by the first device in wireless signals transmitted by the second device.

10. The medium of claim 8, wherein the first device is a mobile device and wherein the second device comprises a sensor configured to determine the plurality of values by sensing the one or more current vital signs for the mammal.

11. The medium of claim 8, wherein presenting the notification comprises:
displaying a visual indication; and/or
playing a sound.

12. The medium of claim 8, wherein determining that the at least one value in the received plurality of values matches a particular notification criteria comprises:
comparing a pattern associated with two or more values in the plurality of values with a particular pattern corresponding to a health-related diagnosis.

13. The medium of claim 8, wherein determining that the at least one value in the received plurality of values matches a particular notification criteria comprises determining:
(a) the at least one value is different than an expected value for the mammal; and/or
(b) a pattern associated with two or more values in the plurality of values is different than an expected pattern for the mammal.

14. The medium of claim 8, wherein the operations further comprises:
prior to determining that the at least one value matches the particular notification criteria:
determining the particular notification criteria based on values for current vital signs of one or more other mammals.

15. The medium of claim 8, wherein the first device is dynamically selected by the second device for transmission of the wireless signals based on an identity of the mammal.

16. The medium of claim 8, wherein the first device is dynamically selected by the second device for transmission of the wireless signals based on the plurality of values.

17. The medium of claim 8, wherein the operations further comprises:
prior to determining that the at least one value matches the particular notification criteria:
identifying a current activity of the mammal; and
dynamically determining the particular notification criteria based on the current activity of the mammal.

18. The medium of claim 8, wherein the operations further comprises:
prior to determining that the at least one value matches the particular notification criteria:
identifying one or more environmental factors corresponding to an environment in which the mammal is currently located; and
dynamically determining the particular notification criteria based on the environmental factors.

19. The medium of claim 8, wherein determining that the at least one value matches the particular notification criteria comprises determining that changes in the vital signs for the mammal do not match trends of changes in vital signs for other mammals.

20. The medium of claim 8, wherein the particular notification criteria is dynamically determined based on a current distance between the first device and the second device.

21. The medium of claim 8, wherein the particular notification criteria is dynamically determined based on a set of one or more mammals that the mammal is currently interacting with.

22. A non-transitory computer readable medium comprising instructions which, when executed by one or more hardware processors, causes performance of operations comprising:
sensing, by a particular device, one or more current vital signs for a mammal;
determining, by the particular device, whether a plurality of values corresponding to the current vital signs for the mammal match a particular notification criteria;
responsive to determining that the plurality of values match the particular notification criteria, wirelessly transmitting the plurality of values to a second device;
responsive to determining that the plurality of values do not match the particular notification criteria, refraining from wirelessly transmitting the plurality of values to the second device.

23. A non-transitory computer readable medium comprising instructions which, when executed by one or more hardware processors, causes performance of operations comprising:
dynamically a notification criteria associated with one or more current vital signs of a mammal;
receiving a plurality of values corresponding to the one or more current vital signs for the mammal;
determining that one or more values in the plurality of values match the notification criteria;
responsive to determining that the one or more values in the plurality of values match the notification criteria;
selecting a particular notification method, from a plurality of notification methods, based on the one or more values in the plurality of values.

24. The medium of claim 23, wherein selecting the particular notification method from the plurality of notification methods comprises selecting a particular recipient from a plurality of recipients for transmission of the notification.

25. The medium of claim 23, wherein selecting the particular notification method from the plurality of notification methods comprises selecting a particular form of communication from a plurality of communication forms for transmission of the notification.

26. The medium of claim 23, wherein selecting the particular notification method from the plurality of notification methods comprises one or more of: an audible notification and a visual notification.

27. The medium of claim 23, wherein selecting the particular notification method from the plurality of notification methods is further based on a location of the mammal.

28. The medium of claim 23, wherein selecting the particular notification method from the plurality of notification methods is further based on a distance between the mammal and one or more other mammals associated with said mammal.

29. The medium of claim 23, wherein selecting the particular notification method from the plurality of notification methods is further based on a distance between the mammal and one or more medical facilities.

30. The medium of claim 23, wherein selecting the particular notification method, from a plurality of notification methods, based on the one or more values in the plurality of values comprises:
identifying a particular threshold, of a plurality of thresholds in the notification criteria, exceeded by the one or more values;
selecting the particular notification method based on the particular threshold.
